(19) United States
(12) Patent Application Publication
(10) Pub. No.: US 2025/0065078 A1
(43) Pub. Date: Feb. 27, 2025

(54) RECONFIGURABLE RIGIDIZING STRUCTURES

(71) Applicant: Neptune Medical Inc., Burlingame, CA (US)

(72) Inventors: Stephen J. MORRIS, Sunnyvale, CA (US); Alexander Q. TILSON, Burlingame, CA (US)

(21) Appl. No.: 18/727,032

(22) PCT Filed: Jan. 4, 2023
(86) PCT No.: PCT/US2023/060086

§ 371 Date: Jul. 5, 2024

Related U.S. Application Data
(60) Provisional application No. 63/296,478, filed on Jan. 4, 2022.

(51) Int. Cl. A61M 25/00

(52) U.S. Cl. CPC A61M 25/0045; A61M 2025/0063

(57) ABSTRACT

Described herein are reconfigurable structures that can transition quickly from a flexible configuration to a rigid configuration core layer, face sheet layers, and an outer sealed layer form the reconfigurable structure. The reconfigurable structures can transition from the flexible configuration to the rigid or stiff configuration, for example, by applying a vacuum or pressure to sealed structure. With the vacuum or pressure removed, the layers can easily shear or move relative to each other. With the vacuum or pressure applied, the layers can transition to a condition in which they exhibit substantially enhanced ability to resist shear, movement, bending, and buckling, thereby stiffening into a rigid configuration.

### CLAIM OF PRIORITY

This patent application claims priority to U.S. Provisional patent application No. 63/296,478, titled “RECONFIGURABLE STRUCTURES” and filed on Jan. 4, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

This application relates to the field of reconfigurable structures that can transition from a flexible to a stiff configuration.

### FIELD OF USE

Cylindrical rigidizing devices (e.g., tubes, rods, etc.) have been described for use in navigating through tortuous anatomy such as (but not limited to), the gastrointestinal tract, the bile and pancreatic ducts, the vascular system (including the heart, peripheral, pulmonary, and neurovasculature), and the airways. Such elongated devices may transition quickly from a flexible configuration (i.e., one that is relaxed, limp, or floppy) to a rigid configuration. These devices may include a plurality of layers, e.g., coiled or reinforced layers, slip layers, braided layers, other rigidizing layers, bladder layers and/or sealing sheaths. The application of vacuum or pressure can cause these devices to transition from a flexible configuration to a rigid configuration, and generally include a tubular configuration, but can also include a rod (solid cylinder) configuration.

Existing non-tubular structures with reconfigurable shape include structures with complex or expensive designs and/or manufacturing methods. For example, a material including sheets of linked structures, such as rings, cubes, and octahedrons, have recently been described. These (typically 3D printed) materials are designed such that they can jam under compression and tension. Other rigidizing materials include shapes controlled by embedding networks of heat responsive liquid crystal elastomers (LCEs) or thin strips of polymer that shrink when heated. These LCEs contain stretchable heating coils that can be charged with electrical current, which heats them up and causes them to contract. As the LCEs contract, they tug at the flexible material into which they were embedded and compressed it into a predesigned solid shape. Particle-jamming stiffening techniques have also been extensively described.

A need exists for a reconfigurable structure with a simple, reliably constructed configuration, which may provide consistent cross-sectional thickness and low material mass. It would also be useful to provide rigidizing and reconfigurable structures that may be used without requiring significant electronics.

### SUMMARY OF THE DISCLOSURE

Described herein are apparatuses (e.g., systems and devices, including structural systems) that may selectively rigidize from a soft, foldable, and flexible sheet configuration that may conform or deform into a desired shape, which can be ‘frozen’ or releasably locked into a structure that is rigid and stiff. Also described herein are methods of using these apparatuses. These apparatuses may be used as medical devices, of for non-medical uses, including, for example, for dwellings, architectural structures or emergency shelters that compact easily and then can quickly be assembled. In general, these apparatuses may be used for any component that would benefit from being transformed from a flexible sheet into a rigid structure.

For example, described herein are rigidizable shell structures, comprising a flexible core layer; a first shear enhancing layer positioned on a first side of the flexible core layer, a first face sheet adjacent to the first shear enhancing layer; a second shear enhancing layer positioned on a second side of the flexible core layer, and a second face sheet adjacent to the second shear enhancing layer, wherein the second side of the flexible core layer is opposite from the first side of the flexible core layer; and an outer cover sealed around the core layer, the first shear enhancing layer, the first face sheet, the second shear enhancing layer and the second face sheet; and an inlet positioned within the outer cover is configured to allow application of pressure within the sealed enclosure, wherein the first face sheet and the second face sheet are configured to shear against the flexible core layer in a first configuration that is flexible without application of pressure, and wherein the first face sheet and the second face sheet are pressure affixed to the core layer in a second configuration that is rigid under the application of pressure.

The pressure may be negative pressure.

In some examples the core layer comprises Nomex or aluminum honeycomb.

In any of these examples multiple cores layers may be utilized. The core layer may comprise foam. The core layer may comprise scored wood (e.g., balsa wood). The core layer may be discontinuous. The core layer may comprise a thickness of about 0.1-10 cm, 0.5-10 cm, 0.1-5 cm, 0.5-5 cm, 0.5-2 cm, 1-5 cm, 1-10 cm, 2-10 cm, 2-8 cm, 2-5 cm, 3-10 cm, 3-8 cm, 3-5 cm, 4-10 cm, 4-8 cm, 4-5 cm, or 5-10 cm. The core layer may have a density of between about 2-20 lb/ft³.

The face sheet may comprise a fiber cloth that is woven or braided. In some examples, the face sheet comprises unidirectional fibers that are intermittently attached to each other. In some examples the fiber cloth comprises a braid or weave angle of about 0-90°, 10-90°, 20-90°, 30-90°, 40-90°, 50-90°, 60-90°, 70-90°, 80-90°, 20-80°, 30-70°, 40-60°, 30-50°, or 40-50°. The braid angle may be the angle between adjacent fibers or bundles of fibers.

The face sheets may comprise a thickness of about 0.1-5 cm, 0.5-5 cm, or 0.5-2 cm. The face sheets may have a coverage of about 30%-70%, such as 40%-60%, e.g., 30%, 40%, 50%, 60%, or 70%. The face sheets may contain tensile elements that have a modulus of elasticity of about 1-400 GPa (e.g., about 10-200 GPa, 50-200 GPa, 50-100 GPa, 60-100 GPa, 70-100 GPa, greater than 100 GPa, or greater than 200 GPa). The face sheets may be comprised of materials that have a density of about 0.1-3 g/cm³, 0.1-2 g/cm³, 0.1-4 g/cm³, 0.1-5 g/cm³, 0.1-6 g/cm³, 0.1-7 g/cm³, 0.1-8 g/cm³, 0.1-9 g/cm³, or 0.1-10 g/cm³. The face sheets may comprise fibers that are rectangular, flat, round, and/or oval. The face sheets may comprise fibers that are plastic or metal. The face sheets may be comprised of monofilaments, or bundles of small fibers, with those fibers up to 1000 fibers per bundle.

The outer cover may comprise an elastomer. The outer cover may have a durometer of about 30 A-80 A, 40-70 A, 50-60 D. The outer cover may comprise a plastic. The outer cover may have a thickness of about 0.0001-1″, 0.001-1″, 0.005-1″, 0.01-1″ 0.05-1″, 0.01-0.5″, 0.5-1″.

In any of these apparatuses, the inlet is attached to tubing.

Also described herein are methods of transitioning a shell structure from a flexible to a stiff configuration, comprising: setting a shell structure into a shape or position, the shell structure comprising a sealed outer layer surrounding a core layer positioned between a first face sheet and a second face sheet, the first and second face sheets configured to shear against the core layer and/or against a shear enhancing layer; applying pressure to the shell structure through an inlet in the sealed outer layer, thereby causing the first and second face sheets to pressure affix to the core layer and causing the shell structure to stiffen in the shape or position.

Any of these methods may include applying pressure to the shell structure by applying negative pressure (e.g., vacuum) to the shell structure. Applying pressure to the shell structure may comprise applying 0.01-14.7 psi negative pressure to the shell structure. Any of these methods may include discontinuing application of pressure, thereby allowing the shell structure to transition back to a flexible configuration.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows an example of a reconfigurable shell structure.

FIG. 2A shows an example of a reconfigurable shell structure being shaped around a cylinder.

FIG. 2B shows the shaped shell structure of FIG. 2A supporting the weight of the cylinder.

FIG. 3A shows an example of a reconfigurable shell structure being shaped around an end of a cylinder.

FIG. 3B shows the shaped shell structure of FIG. 3A supporting the weight of the cylinder.

FIG. 4A shows an example of a reconfigurable shell structure being shaped around a rod.

FIG. 4B shows the reconfigurable shell structure of FIG. 4B with the rod removed and the structure retaining the tubular shape.

FIG. 4C shows the reconfigurable shell structure of FIG. 4C supporting the weight of a cylinder.

FIG. 5A schematically illustrates a cross-section through one example of a reconfigurable shell structure in the relaxed (un-rigidized, highly flexible) configuration.

FIG. 5B schematically illustrates a cross-section through the reconfigurable shell of FIG. 5A in the rigidized configuration.

FIG. 5C schematically illustrates a cross-section through another example of a reconfigurable shell in a relaxed (un-rigidized, highly flexible) configuration.

FIG. 6A illustrates a cross-section through another example of a reconfigurable shell structure in the relaxed (un-rigidized, highly flexible) configuration similar to that shown in FIG. 5A.

FIG. 6B is a cross-section through another example of a reconfigurable shell structure in the relaxed (un-rigidized, highly flexible) configuration; the structure shown in FIG. 6B is formed of stacked layers of stacked core layers.

FIG. 7A shows an example of a braided plastic monofilament material layer that can be used as a face sheet in a reconfigurable shell structure.

FIG. 7B shows an example of a woven fiber cloth material layer that can be used as a face sheet in a reconfigurable shell structure.

FIGS. 8A and 8B show embodiments of material layers that can be used as a core layer in a reconfigurable shell structure.

### DETAILED DESCRIPTION

Described herein are apparatuses (e.g., systems and devices), including planar sheets of material that can be controllably and rapidly transitioned from a highly flexible configuration (i.e., one that is relaxed, limp, or floppy) to a highly rigid configuration (i.e., one that is stiff and/or holds the shape it is in when it is rigidized) by the application of negative pressure (e.g., vacuum). These apparatuses may be referred to as rigidizable sheets and/or as rigidizable shell structures.

The structures described herein may rigidize quickly (e.g., almost instantaneously, e.g., within less than 1 second, less than 0.5 seconds, less than 0.3 seconds, less than 0.2 seconds, less than 0.1 seconds, etc.). Larger structures may take longer to rigidize as their volume is larger. They could be configured to rigidize quickly by including a plurality of pressure (e.g., suction) ports that are distributed within the structure.

In general, any of these structures may transition without shrinking appreciably in the direction of the planar surface.

These apparatuses may include stacks of layers that are enclosed by a sealed region into which vacuum may be applied. The layers include one or more core layers that are surrounded on both sides by a shear enhancing layer and on the outside of the stack by face sheets. The shear enhancing layers may be optional and may not be included in some examples; for example, the face sheets could directly contact the core. Layers are stacked onto each other and enclosed within the sealed or sealable region that is connected to one or more ports to which a vacuum (negative pressure) may be applied. In general, the layers may be freely slidable and flexible relative to each other in the relaxed configuration, in which little or no vacuum is applied. When negative pressure (e.g., vacuum) is applied, the stacks may laminate together and dramatically increase the stiffness of the assembly. The stiffness may be regulated by regulating the magnitude of the negative pressure.

Furthermore, the stiffness can be varied by modulating the core, including increasing or decreasing the core thickness, the number of core layers, the properties of the face sheets, and/or the properties of the shear enhancing layer. For example, increasing the thickness of the core layer(s) (or increasing the total thickness of all of the core layers) typically allows the structure to achieve a greater final stiffness, while decreasing the core thickness (or decreasing the total thickness of all of the core layers) typically decreases the final stiffness for a comparable pressure. The core layer may be divided up into multiple core layers that are separated by a shear enhancement layer (or multiple shear enhancement layers). Adding additional core layers (and thereby, overall thickness) may dramatically increase the final stiffness that can be achieved in the rigidized configuration, without dramatically increasing the flexibility in the un-rigidized configuration. Thus, any number of core layers may be used. In general, increasing the thickness of the core layer(s) may increase the ultimate stiffness in the rigidized configuration for a comparable negative pressure. The core layer (or region) may be divided into multiple core layers. For a given thickness, having more (but thinner) core layers may increase flexibility without significantly compromising rigidized stiffness.

In the un-rigidized configuration the rigidizable shell structures described herein may be highly drapeable and may readily confirm to a three-dimensional surface or shape that may be rigidized by the application of negative pressure.

Any appropriate core sheet or layer may be used. In general, the core layer or sheet may be highly flexible in bending and has a well-defined thickness. The core provides a cross sectional height that may be set or fixed. The core can may have a solid surface or a porous or honeycomb structure and may be formed of any appropriate material that allows flexibility in bending while resisting compression. For example, the core may be formed of one or more multiple materials, and may be formed into a pliable structure, including but not limited to a honeycomb structure (e.g., Nomex™ or aluminum), foam (e.g., a sheet or scored with a back-supporting matrix), or balsa wood (again, typically scored, with a back-supporting matrix).

The core may be formed of multiple layers; as mentioned each core layer may be separated from an adjacent core layer by a shear enhancing layer. Stacking multiple core layers may enhance baseline flexibility; the ultimate stiffness may be related to the aggregate thickness but dividing the core into constituent layers may allow interlaminar core layer shear, which enhances flexibility.

The face sheets may be generally relatively lightweight and have a high tensile stiffness and exhibit high shear stiffness but may be formed into a pliable layer. For example, face sheets may be formed of a woven, knitted or knit layer or material. For example, the face sheets may be formed of a fiber cloth (without a rigidizing resin). This cloth can be, for example, woven or braided, and it can be formed of a fiber, a high strength fiber, a plastic, or a metal, or some combination of these, e.g., a fiber/epoxy laminate, aluminum or stainless steel. Face sheets may be formed of fibers of material having an angle, thickness, open area, modulus, fiber diameter and/or weave pattern that may be modulated or selected to maximize the flexibility in the relaxed configuration of the apparatus while maximizing the rigidity in the rigidized configuration(s). In general, the face sheets may be highly flexible in the relaxed configuration, but when suction is applied, the face sheets may be laminated against the core layer(s) to form an extremely rigid structure.

Any appropriate shear enhancing layer may be used which may transfer loads from the face sheets to the core(s), such that rigidized stiffness is enhanced. For example, the shear enhancing layers may be formed of an elastomeric material having a variety of different durometers and thicknesses, which may be set to optimizes flexibility in the relaxed configuration while maximizing stiffness in the rigidized configuration.

The sealed structure (e.g., sealable enclosure) may be formed of a flexible and air-impermeable material, such as a plastic or an elastomeric material. The sealed or sealable enclosure may hold the component layers inside the sealed structure so that the application of vacuum (negative pressure), causes the face sheets to be vacuumed against the core so that they provide inter-fiber shear stabilization of the face sheets, while pressure-affixing them to the core. When the pressure (vacuum) is released, the fibers forming the face sheet are free to shear relative to each other, the face sheets are no longer vacuumed to the core, and the unit reverts to a hyper-flexible mode.

Thus, a plurality of layers (e.g., a core layer, face sheet layers, shear enhancing layer(s), and a bladder layer and/or sealing outer layer) can together form the reconfigurable structures. The reconfigurable structures can transition from the flexible configuration to the rigid or stiff configuration, for example, by applying a vacuum or pressure to sealed structure. With the vacuum or pressure removed, the layers can easily shear or move relative to each other. With the vacuum or pressure applied, the layers can transition to a condition in which they exhibit substantially enhanced ability to resist shear, movement, bending, and buckling, thereby providing rigidization.

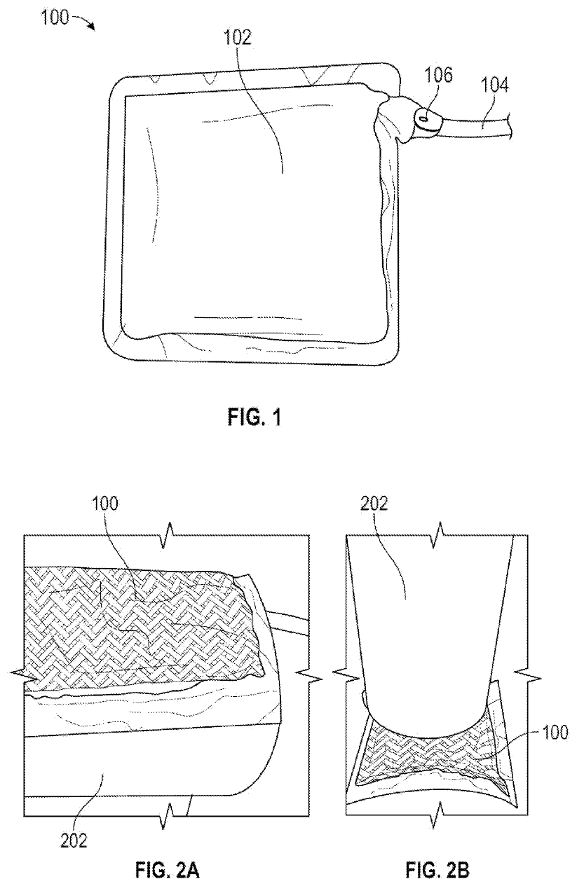

FIG. 1 shows an embodiment of a reconfigurable structure 100. The structure 100 comprises a sealed region 102. The sealed region comprises an inlet 106 that is fluidly connected to a vacuum/pressure line 104. Without the application of pressure or vacuum, the structure 100 is flexible and is able to be bent or curved into various different configurations. The example shown in FIG. 1 shows a single port or inlet to the vacuum/pressure line 104; in practice multiple inlet pressure lines and/or outlet lines may be included. For example, a single apparatus may include multiple regions that are selectively and separable rigidizable. The inlet and/or outlet lines may be valved or otherwise sealable. In FIG. 1 the outer perimeter of the apparatus is not part of the rigidizable structure. In some examples other subregions of the apparatus may not be rigidizable but may remain flexible. In some examples all of the apparatus may be rigidizable.

Referring now to FIG. 2A, an example of a reconfigurable apparatus (e.g., structure 100, which is similar to that shown in FIG. 1) is shown curved around a cylinder 202. Without the application of pressure or vacuum, the apparatus (e.g., structure 100) is flexible enough to curve in this manner. After the application of pressure or vacuum while curved around the cylinder, the structure 100 is stiffened into its assumed shape, as shown by the reconfigured shape in FIG. 2B. FIG. 2B also shows that the stiffened structure 100 is strong enough to withstand the weight of the cylinder.

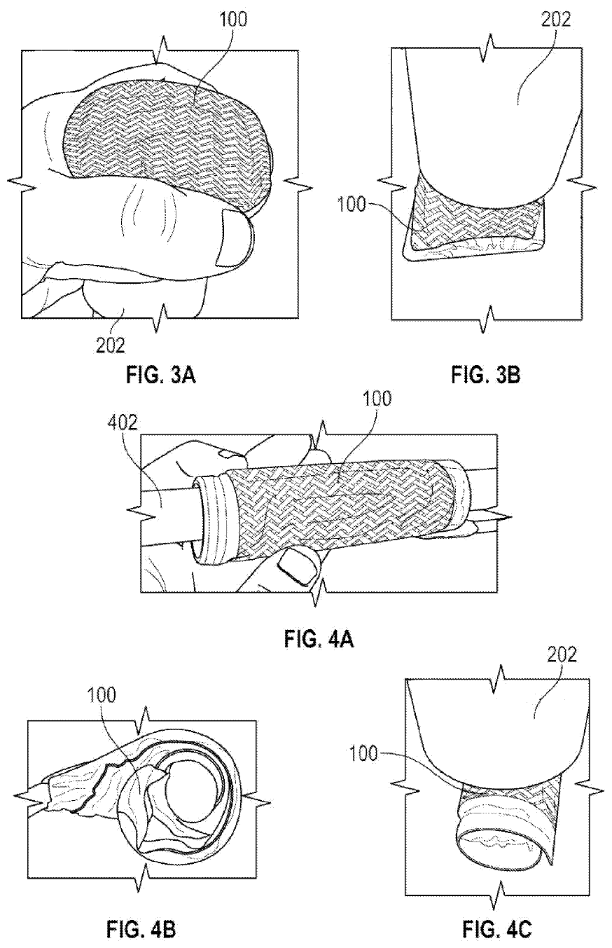

FIG. 3A shows the reconfigurable structure 100 wrapped around an end of a cylinder 202. Without the application of pressure or vacuum, the structure 100 is flexible enough to curve in this manner. After the application of pressure or vacuum while curved around the cylinder, the structure 100 is stiffened into its assumed shape, as shown by the reconfigured shape of FIG. 3B. FIG. 3B also shows that the stiffened structure 100 is strong enough to withstand the weight of the cylinder.

Referring to FIG. 4A, the reconfigurable structure 100 is shown wrapped or draped around a rod 402. Without the application of pressure or vacuum, the structure 100 is flexible enough to curve in this manner. After the application of pressure or vacuum while curved around the rod, the structure 100 is stiffened into its assumed shape, as shown by the reconfigured shape of FIG. 4B. FIG. 4C shows that the stiffened structure 100 is strong enough to withstand the weight of the cylinder 202.

Ceasing the application of pressure (e.g. turning off the vacuum and/or venting to air) causes the stiffened structure to return or move towards its flexible configuration.

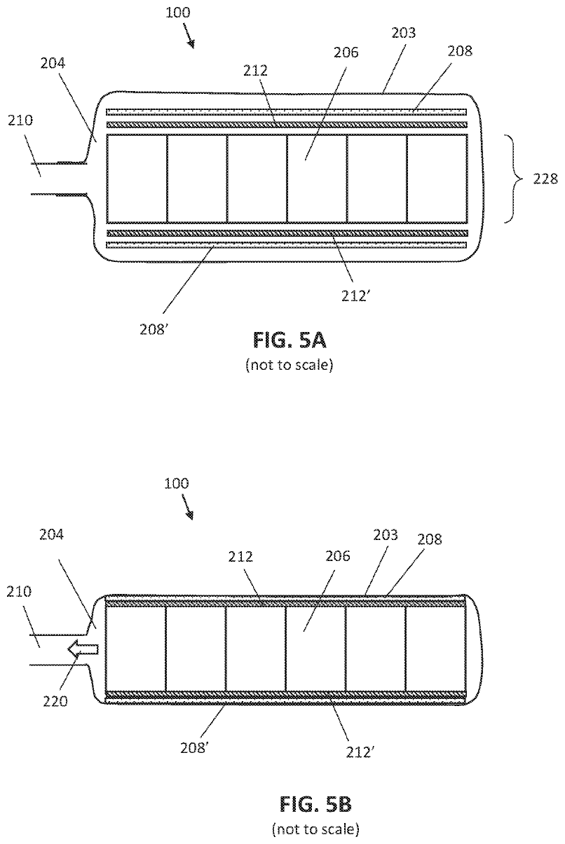

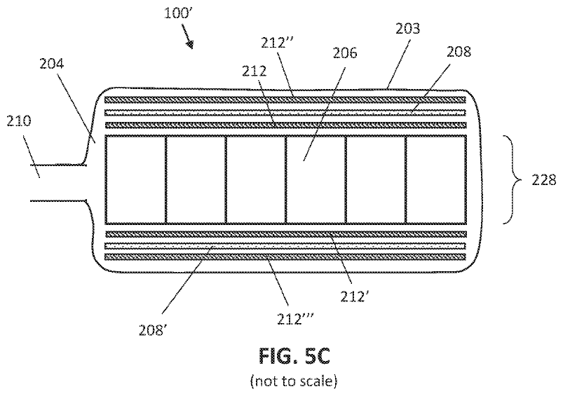

FIG. 5A shows an example of a cross-sectional schematic view of a reconfigurable structure 100. In this example (not to scale), the structure 100 comprises a sealed enclosed region 204. The sealed enclosed region 204 encloses a single core 206 that is flanked on the upper surface and the lower surface by inner-fiber shear stabilizing layers 212, 212′ that are adjacent to outer face sheet layers 208, 208′. The enclosed region may be enclosed by, e.g., an elastomeric or plastic material 203, and may be referred to as the outer sealing container. A vacuum/pressure line 210 is fluidly connected to the sealed region 204. The vacuum pressure line may be supported to prevent collapse when suction is applied. In some examples the suction (negative pressure) is applied from the end of the apparatus, as shown. Alternatively or additionally, such may be distributed within the structure and may be applied at more than one location, and/or at a central location.

FIG. 5B schematically illustrates the apparatus of claim 5A in a rigidized configuration in which negative pressure (vacuum) is applied. In this example the negative pressure laminates the outer face sheets 208, 208′ against the inter-fiber shear stabilization sheets 212, 212′ and the core layer 206. The core may be relatively incompressible in that axis. The spacing between layers in FIG. 5A is exaggerated, as the layers (including the outer sealing chamber) may lay flat, though loosely, against each other in the unrigidized configuration.

FIG. 5C shows another cross-sectional schematic view of another example of a reconfigurable structure 100′ including a sealed enclosed region 204 that encloses a single core 206 that is flanked on the upper surface and the lower surface by inner-fiber shear stabilizing layers 212, 212′ that are adjacent to outer face sheet layers 208, 208′. In this example a third and fourth shear stabilizing layer 212″, 212″′ are also included outside of the outer face sheet layers. In any of the apparatuses described the additional shear stabilizing layers 212″, 212″′ could exist on both sides of the face sheet 208, 208′. This may provide enhanced shear properties with the face sheet, thereby providing enhanced rigidization.

In any of the apparatuses described herein the shear stabilizing layer 212, 212′ could be bonded to core 206 (or to respective cores). This may still provide shear enhancing properties relative to the face sheets. It may be more firmly attached and provide enhanced rigidization, while still allowing the core to have flexibility, because the shear stabilizing layers could be an elastomer.

Any of the apparatuses described herein may include (e.g., between the inside of the outer sealing container, e.g., bag 203, and the outer layer of the composite stack of layers, e.g., the outer face sheet 208 or an outer shear layer 212″), a breathable material, such as a cloth or a random orientation fibered ‘breather’, that may enhance air evacuation from the apparatus.

As mentioned, the core layer may provide a well-defined thickness or cross-sectional height to the reconfigurable structure, while also providing flexibility. In some embodiments, the core layer comprises a material that is discontinuous along its surface. Discontinuity can refer to the material comprising scores along its surface. Discontinuity can also refer to a material configured, at least in part, as a mesh, web, or net, or otherwise comprising connected strands of material (e.g., honeycomb).

In some embodiments, the core layer comprises a contact area percentage of about 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 10-90%, 20-80%, 30-70%, 40-60%, 45-55%, etc. In some examples the core contact area may be low (e.g., less than 15%, less than 10%, less than 8%, less than 7%, less than 6%, less than 5%, less than 4%, less than 3%, less than 2%, less than 1%, etc.)

In some embodiments, each core layer comprises a thickness of about 0.1-10 cm, 0.5-10 cm, 0.1-5 cm, 0.5-5 cm, 0.5-2 cm, 1-5 cm, 1-10 cm, 2-10 cm, 2-8 cm, 2-5 cm, 3-10 cm, 3-8 cm, 3-5 cm, 4-10 cm, 4-8 cm, 4-5 cm, 5-10 cm, etc. Multiple core layers may be used and separated by shear stabilization layer(s). The overall core thickness may be large. For example, the overall core (or core region) may have a thickness that is between 0.1 cm and 50 cm (e.g., between 0.1 cm and 40 cm, between 1 cm and 35 cm, between 1 cm and 31 cm, between 1 cm and 25 cm, between 1 cm and 16 cm, etc.).

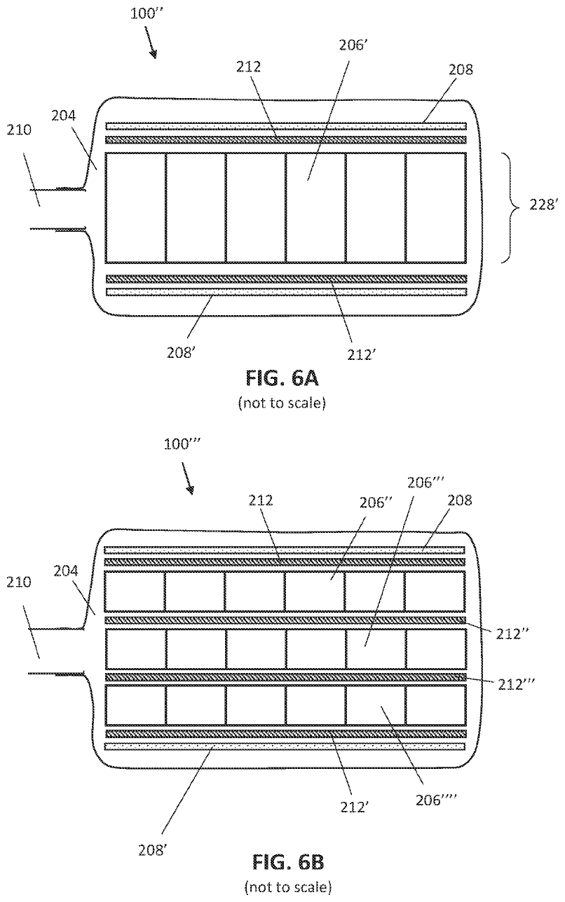

For example, FIG. 6A shows an example of an apparatus 100″ similar to that shown in FIG. 5A, including a core 206′, flanked by a pair of shear stabilizing layers 212, 212′ and face sheets 208, 208′. In this example the core 206 has a greater height 228′ than the height 228 of the core in FIGS. 5A and 5B. The greater height may result in a greater stiffness when negative pressure is applied.

FIG. 6B shows another example of a schematic of a rigidizable shell structure 100″′ as described herein. In FIG. 6B the core includes multiple core layers 206″, 206″′, 206″ ″ that are each separated by a shear stabilizing layers 212″, 212″′. The total core thickness of the example shown in FIG. 6B is similar to that shown in FIG. 6A, and the ultimate stiffness under similar negative pressure may be approximately equivalent; however the baseline flexibility of the apparatus of FIG. 6B, having multiple core layers may be significantly higher.

In some embodiments, the core layer comprises a density of between about 1 and 30 lb/ft³ (e.g., 2-20 lb/ft³, 2-18 lb/ft³, 2-15 lb/ft³, etc.). In general, the core is configured to be flexible in bending but to be incompressible and to provide a defined height though which shear loads are transmitted. The core layer can comprise one or more of a variety of materials. In some embodiments, the core layer comprises Nomex or aluminum (e.g., Nomex or aluminum honeycomb, as shown in FIG. 8A). As mentioned, in some embodiments, the core layer comprises foam. The foam can be at least partially scored or can comprise an unscored sheet. In some scored embodiments, the foam comprises a back-supporting matrix. In some embodiments, the core layer comprises balsa wood. The balsa wood can be at least partially scored (FIG. 8B) or can comprise an unscored sheet. In some scored embodiments, the balsa wood comprises a back-supporting matrix.

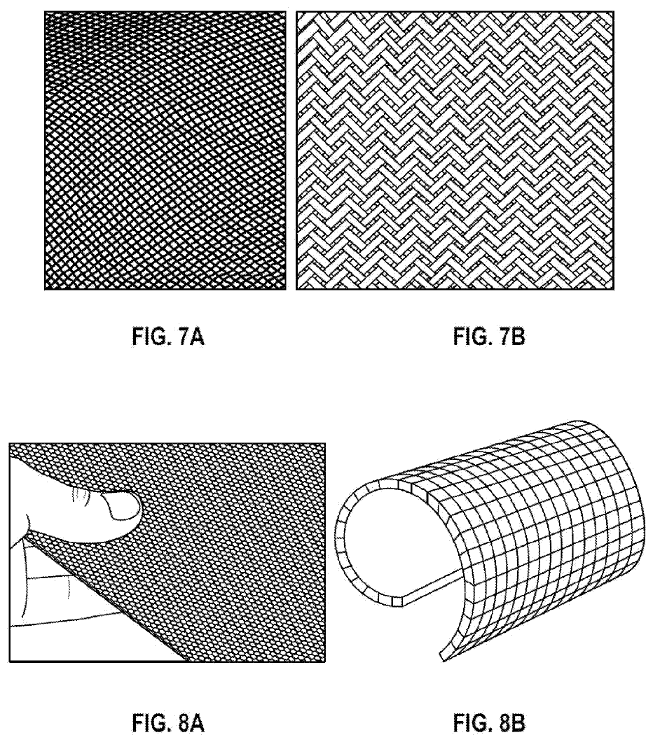

As mentioned above, the face sheet may comprise a material that can shear relative to adjacent components, allowing the structure to be deformable. For example, the face sheet can comprise a fiber cloth. The cloth can be woven (see, e.g., FIG. 7B) or braided (e.g., as braided plastic as shown in FIG. 7A). The cloth may comprise multiple layers of unidirectional fibers that are intermittently attached to each other. The fiber in the cloth can comprise a high-strength and high axial stiffness fiber (e.g., carbon, Kevlar™, Technora™ fiberglass, Dyneema™, Vectran™, etc.).

In some embodiments, the face sheet cloth can have a weave or braid angle of fibers relative to a longitudinal axis of the structure of about 0-90°, 10-90°, 20-90°, 30-90°, 40-90°, 50-90°, 60-90°, 70-90°, 80-90°, 20-80°, 30-70°, 40-60°, 30-50°, 40-50°, etc.

In some embodiments, the face sheets comprise a thickness of about 0.1-5 cm, 0.5-5 cm, 0.5-2 cm, 1-5 cm, 2-5 cm, 3-5 cm, 4-5 cm, etc.

In some embodiments, the face sheets comprise a coverage of 30%-70%, such as 40%-60%, e.g., 30%, 40%, 50%, 60%, or 70%, where the coverage area is the percentage of an underlying surface that is covered or obstructed by the fibers of the cloth.

In some embodiments, the face sheets contain tensile elements that have a modulus of elasticity of about 1-400 GPA, 10-200 GPa, 50-200 GPa, 50-100 GPa, 60-100 GPa, 70-100 GPa, greater than 100 GPa, greater than 200 GPa, etc. In general, the tensile strength and stiffness of the fibers may be relatively high, although the fibers or filaments may move or shear relative to each other.

In some embodiments, the face sheet comprises a lightweight material, comprising a low density. The density can be less than about 1 g/cm³, between about 0.1-3 g/cm³, 0.1-2 g/cm³, 0.1-4 g/cm³, 0.1-5 g/cm³, 0.1-6 g/cm³, 0.1-7 g/cm³, 0.1-8 g/cm³, 0.1-9 g/cm³, 0.

1-10 g/cm³, etc. The fibers can be rectangular/flat (e.g., with a long edge of 0.001″-0.060″, such as 0.005″, 0.007″, 0.010″, or 0.012″, and a short edge of 0.0003″-0.030″, such as 0.001″, 0.002″, or 0.003″), round (e.g., with a diameter of 0.001″-0.020″, such as 0.005″, 0.01″, or 0.012″), or oval. In some embodiments, some of the fibers can be flat and some of the fibers can be round.

In some embodiments, the fibers can be made of metal filaments (e.g., stainless steel, aluminum, nitinol, tungsten, or titanium), plastic (nylon, polyethylene terephthalate, PEEK, polyetherimide), or high strength and high axial stiffness fiber (e.g., carbon, Kevlar™ Technora™, aramids, fiberglass, Dyneema™ or UHMWPE, or liquid crystal polymers such as Vectran™). In some embodiments, the fibers can be made of a multi-layer composite, such as a metal core with a thin elastomeric coating.

The face sheets may be comprised of monofilaments, or bundles of small fibers, with those fibers up to 1000 fibers per bundle. A higher number of strands may advantageously help stiffen the face sheet due to the increased interaction between fibers.

The core and face sheets are held within a sealed region. The sealed region can comprise a flexible material that is fluid impermeable. The sealed region can be configured to move radially inward when a vacuum is applied to pull down against the inner layers and conform onto the surface(s) thereof. The sealed region boundary layer can be soft and atraumatic and can be sealed at both ends to create a vacuum-tight chamber. The sealed region boundary layer can comprise a plastic. The sealed region boundary layer can be elastomeric, e.g., made of urethane.

In some embodiments, the entire structure comprises the sealed region. In some embodiments, the sealed region only makes up a portion or portions of the structure.

The hardness of the sealed region boundary layer can be, for example, 30 A-80 A, 40-70 A, 50-60 D, etc.

The thickness of the sealed region boundary layer can be about 0.0001-1″, 0.001-1″, 0.005-1″, 0.01-1″ 0.05-1″, 0.01-0.5″, 0.5-1″, etc.

In some embodiments, the sealed region boundary layer can be plastic, including, for example, polyethylene, nylon, or PEEK.

A vacuum/pressure line 210 is fluidly connected to the sealed region 204. The vacuum/pressure line 210 can comprise tubing (e.g., plastic or elastomeric tubing). In some embodiments, the line 210 is configured to apply between minimal to full atmospheric vacuum (e.g., approximately 14.7 psi).

The face sheets are selectively adhered or attached to the core through the application of pressure (e.g., negative pressure). This selective attachment/detachment of the face sheets to the core is what allows the structure to toggle between the stiff and flexible configurations.

The stiffness of the structure can be varied by modulating the core (e.g., thickness of the core, drapeability, contact area percentage, etc.). The stiffness can also be adjusted by modulating the face sheets (e.g., material, angle, thickness, open area, modulus, fiber diameter, weave pattern, etc.). The stiffness can also be adjusted by modulating the sealed region properties (e.g., modulus, durometer, thickness, etc.). The stiffness of the structure can be varied by adjusting the pressure or vacuum level.

An exemplary method of use comprises setting the reconfigurable structure into a position or shape. Vacuum can then be applied to the system. As the face sheets are vacuumed against the core, they provide inter-fiber shear stabilization of the face sheets, while pressure affixing them to the core. When the pressure is released, the face sheets are no longer vacuumed to the core, and the fibers are free to shear relative to each other, causing the system to revert to its flexible configuration.

The structures disclosed herein can advantageously be stowed in a first condition (e.g., flat or rolled) and then be rapidly transitioned into a load-bearing structure capable of assuming a wide variety of shapes.

The structures described herein can come in any number of shapes (e.g., square, circular, ovular, rectangular, etc.). The structures can be used as medical devices. For example, they can be used for elements that need to enter the body through a small orifice and then expand out inside the body to become a larger plate or structure that can be used to push, move, or reposition anatomy. As another example, the structure can be used for exoskeletons or casts.

The structures described herein can also be used in non-medical applications. For example, the structures can be used for dwellings, architectural structures, or emergency shelters that compact easily and can be quickly assembled.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A rigidizable shell structure, comprising
a flexible core layer;
a first face sheet positioned on a first side of the flexible core layer;
a second face sheet positioned on a second side of the flexible core layer, the second side opposite from the first side of the flexible core layer; and
an outer layer surrounding and forming a seal around the flexible core layer, the first face sheet, and the second face sheet;
an inlet positioned within the outer layer and configured to allow application of pressure therethrough,
wherein the first face sheet and the second face sheet are configured to shear against the flexible core layer in a first configuration that is flexible without application of pressure from the inlet, and wherein the first face sheet and the second face sheet are pressure affixed to the flexible core layer in a second configuration that is rigid under the application of pressure from the inlet.

2. The structure of claim 1, wherein the pressure is negative pressure.

3. The structure of claim 1, wherein the flexible core layer comprises Nomex™ or aluminum honeycomb.

4. The structure of claim 1, in which a shear enhancing layer is utilized.

5. The structure of claim 1, in which multiple cores layers are utilized.

6. The structure of claim 1, wherein the flexible core layer comprises foam.

7. The structure of claim 1, wherein the flexible core layer comprises scored balsa wood.

8. The structure of claim 1, wherein the flexible core layer is discontinuous.

9. The structure of claim 1, wherein the flexible core layer comprises a thickness of about 0.1-10 cm, 0.5-10 cm, 0.1-5 cm, 0.5-5 cm, 0.5-2 cm, 1-5 cm, 1-10 cm, 2-10 cm, 2-8 cm, 2-5 cm, 3-10 cm, 3-8 cm, 3-5 cm, 4-10 cm, 4-8 cm, 4-5 cm, or 5-10 cm.

10. The structure of claim 1, wherein the first face sheet comprises a fiber cloth.

11. The structure of claim 10, wherein the fiber cloth is woven or braided.

12. The structure of claim 1, wherein the first and second face sheets comprise a thickness of about 0.1-5 cm, 0.5-5 cm, or 0.5-2 cm.

13. The structure of claim 1, wherein the face sheets comprise a coverage of about 30%-70%, such as 40%-60%, e.g., 30%, 40%, 50%, 60%, or 70%.

14. The structure of claim 1, wherein the first and second face sheets comprise a density of about 1-3 g/cm³, 1-2 g/cm³, 1-4 g/cm³, 1-5 g/cm³, 1-6 g/cm³, 1-7 g/cm³, 1-8 g/cm³, 1-9 g/cm³, or 1-10 g/cm³.

15. The structure of claim 1, wherein the first and second face sheets comprise fibers that are rectangular, flat, round, and/or oval.

16. The structure of claim 1, wherein the first and second face sheets comprise fibers that are plastic or metal.

17. The structure of claim 1, wherein the outer layer comprises an elastomer.

18. The structure of claim 1, wherein the outer layer comprises a plastic.

19. The structure of claim 1, wherein the outer layer comprises a hardness of about 30 A-80 A or about 50-60 D.

20. The structure of claim 1, wherein the outer layer comprises a thickness of about 0.0001-1″, 0.001-1″, 0.005-1″, 0.01-1″ 0.05-1″, 0.01-0.5″, 0.5-1″.

21. The structure of claim 1, wherein the inlet is attached to tubing.

22. A rigidizable shell structure, comprising
a flexible core layer;
a first sheet positioned on a first side of the flexible core layer;
a second sheet positioned on a second side of the flexible core layer that is opposite from the first side of the flexible core layer; and
an outer layer surrounding and forming a seal around the flexible core layer, the first sheet, and the second sheet;
an inlet positioned within the outer layer and configured to allow application of pressure therethrough,
wherein the first sheet and the second sheet shear against the flexible core layer in a first configuration that is flexible, without applying pressure through the inlet, and wherein the first sheet and the second sheet are rigidly affixed to the flexible core layer in a second configuration that is rigid under the application of pressure through the inlet.

23. A method of transitioning a shell structure from a flexible to a stiff configuration, comprising:
setting a shell structure into a shape or position, the shell structure comprising a sealed outer layer surrounding a core layer positioned between a first face sheet and a second face sheet, the first and second face sheets configured to shear against the core layer;
applying pressure to the shell structure through an inlet in the sealed outer layer, thereby causing the first and second face sheets to pressure affix to the core layer and causing the shell structure to stiffen in the shape or position.

24. The method of claim 23, wherein applying pressure to the shell structure comprises applying negative pressure to the shell structure.

25. The method of claim 23, wherein applying pressure to the shell structure comprises applying between 0.01-14.7 psi negative pressure to the shell structure.

26. The method of claim 23, further comprising discontinuing application of pressure, thereby allowing the shell structure to transition back to a flexible configuration.
